(19) United States
(12) Patent Application Publication
(10) Pub. No.: US 2007/0026402 A1
(43) Pub. Date: Feb. 1, 2007

(54) Genetic marker of response to atypical antipsychotics and antidepressants methods for use thereof

(76) Inventors: Ernest P. Noble, Laguna Beach, CA (US); Rose M. Young, Queensland (AU); Bruce R. Lawford, Queensland (AU)

Correspondence Address: KAREN S. CANADY;CANADY & LORTZ LLP, COMMERCE PLAZA, 11340 WEST OLYMPIC BLVD., SUITE 275, LOS ANGELES, CA, 90064, US

(21) Appl. No.: 10/563,077

(22) PCT Filed: Jul. 8, 2004
(86) PCT No.: PCT/US04/22064

§ 371 Date: Dec. 29, 2005

Related U.S. Application Data
(60) Provisional application No. 60/485,482, filed on Jul. 8, 2003.

(51) Int. Cl. C12Q 1/68

(52) U.S. Cl. 435/6

(57) ABSTRACT

The invention provides methods of identifying candidate psychiatric patients, or patients with movement disorder, for treatment with receptor site or increases density of D2 dopamine receptors. The method comprises determining a patient's DRD2 genotype. Patients having the Taq1A (A1) allele (A1+ allelic status) are candidates for treatment with high dose, high binding antipsychotics and/or SSRIs that influence D2 receptor density. Patients lacking the Taq1A allele (A1− allelic status) are candidates for treatment with low dose, low binding atypical antipsychotics, and are not likely to respond well to these SSRIs.

This application claims the benefit of U.S. provisional application No. 60/485,482, filed Jul. 8, 2003, the entire contents of which are hereby incorporated by reference into this application.

Throughout this application various publications are referenced. The disclosures of these publications in their entireties are hereby incorporated by reference into this application in order to more fully describe the state of the art to which this invention pertains.

### TECHNICAL FIELD OF THE INVENTION

The invention relates to a genetic marker of response to antipsychotic and antidepressant medications and methods of using such marker to guide the selection of an appropriate and effective treatment strategy. More particularly, the invention identifies genotypes associated with responsiveness to medications that act at D2 dopamine receptors and that influence D2 dopamine receptor density.

### BACKGROUND OF THE INVENTION

The treatment of patients with antipsychotic medications is hindered by side-effects and complications that cause considerable distress and result in low adherence to medications. Older, “typical” antipsychotics bind tightly to the D2 dopamine receptor (DRD2) and some patients experience poor response to treatment with these medications. Currently used medications, known as atypical antipsychotics, bind to the D2dopamine receptor with varying strength. While the atypical medications represent a significant advance in the treatment of psychosis, there are no guidelines to match patients to medication type to both maximize clinical response and to minimize the likelihood of harmful adverse effects, such as extrapyramidal movement symptoms and diabetes.

Similarly, response to the class of antidepressant medications known as selective serotonin reuptake inhibitors (SSRI) has been variable. SSRIs are an effective treatment for a wide variety of psychiatric disorders for many patients, but some patients do not respond to medication. There remains a need for predictors of patient response to assist clinicians in the selection of antidepressant as well as antipsychotic medications.

### SUMMARY OF THE INVENTION

The invention provides methods of identifying candidate psychiatric patients or patients with movement disorder for treatment with medication that acts at the D2 dopamine receptor. The method comprises determining a patient's D2 dopamine receptor DRD2) genotype. Patients having the Taq1A (A1) allele (A1+ allelic status) are candidates for treatment with high dose of high D2 dopamine receptor binding antipsychotics and/or SSRIs that influence D2 dopamine receptor density. Patients lacking the Taq1A allele (A1− allelic status) are not likely to respond well to these SSRIs, and are candidates for treatment with lowdose of low D2 dopamine receptor binding or low dose high D2 dopamine receptor binding atypical antipsychotics.

In one embodiment, the psychiatric patient suffers from schizophrenia. In another embodiment, the patient suffers from post-traumatic stress disorder (PTSD), depression, social anxiety or mixed anxiety and depressive states. In a third embodiment, the patient suffers from a movement disorder, such as Parkinson's Disease. It is accepted psychiatric practice to treat a schizophrenic or other psychotic patient with either a high binding antipsychotic, examples of which include Risperidone, Flupenthixol, Fluphenazine decanoate, Zuclopenthixol, Haloperidol, Thiondazine, Thiothixene or Trofluperazine, or a low binding atypical antipsychotic, such as Olanzapine or Clozapine. Patients suffering from PTSD, depression, social anxiety or mixed anxiety and depressive states are typically treated with an SSRI, such as Paroxetine or Halopram.

### BREIF DESCRIPTION OF THE DRAWINGS

FIG. 1 is a bar graph depicting Physician's Examination (ESRS) Parkinsonism Scores of subjects in the four different allele/dosage groups. There was a significant allele by dose interaction (p=0.028)

FIG. 2 is a bar graph showing ESRS Parkinsonism, Dystonia and Dyskinesia: Questionnaire and Behavioral scale (Physician or Nurse) scores for A1 subjects treated with high and low dose Risperidone. Low dose A1 subjects displayed significantly worse symptoms (mean score=4.07, s.d.=2.80) than A1 subjects given high doses of Risperidone(mean score=1.27, s.d.=1.62).(df=1,24, F=8.8, p=0.0067). A1 patients receiving below median doses of Risperidone(A1 Low Dose) had significantly worse symptoms of Parkinsonism, Dystonia and Dyskinesia than A1 patients treated with median dose or above. (p=0.0067).

FIG. 3 is a bar graph that shows prolactin levels across medication groups.

FIG. 4 is a bar graph depicting baseline GHQ total scores and GHQ subscale scores of 63 PTSD patients who entered paroxetine treatment based on the presence or absence of the DRD2 allele. (a) P=0.040; (b) P=0.046; (c) P1=0.033; (d) P=0.011.

FIG. 5 is a bar graph showing results of intention-to-treatment analysis of GHQ subscale scores at baseline and following paroxetine treatment based on the presence or absence of the DRD2 allele. (a) P=0.014.

FIGS. 6A-6D show results of intention-to-treatment analysis of GHQ subscale scores at baseline and following paroxetine treatment based on the presence or absence of the DRD2 allele. (a) P=0.009; (b) P=0.033; (c) P=0.031; (d) P=0.026.

FIG. 7 is a bar graph comparing mean Mississippi scores of Cluster1 and Cluster3 (Cluster1 vs. Cluster 3, F(1,45)=18.75, p<0.0001).

### DETAILED DESCRIPTION OF THE INVENTION

The invention is based on the discovery that Taq1A allelic status is associated with response to medications that act at the D2 dopamine receptor (DRD2) as well as response to selective serotonin reuptake inhibitors (SSRIs). The invention provides an advance in the treatment of patients with psychiatric disorders such as psychosis and depression, by providing a means to identify those patients likely to respond favorably to different types of antidepressant and antipsychotic medications. This allows clinicians to maximize response to treatment, while minimizing the likelihood of adverse side effects.

Definitions

All scientific and technical terms used in this application have meanings commonly used in the art unless otherwise specified. As used in this application, the following words or phrases have the meanings specified.

As used herein, “high dose” of medication means more than the chlorpromazine equivalent per kilogram (kg) of body weight (CPZEK) of about 10. (Given an average adult patient body weight of 70 kg.) One mg risperidone is equipotent to 100 mg chlorpromazine, 100 mg thioridazine, or 2 mg haloperidol. For example, a high dose of risperidone is about 6 mg/day or more for an adult patient.

As used herein, “high D2 dopamine receptor binding” or “high binding” antipsychotics means having an affinity for the D2 dopamine receptor exhibiting a Kᵢ of less than 10 nM, as measured by in vitro radioligand binding (See, e.g., Levant, 1997, Pharmacological Reviews, 49(3):231-252). This class of antipsychotic medications is often referred to in the art as “typical” antipsychotics. Representative examples include rispetidone (resperidone), flupenthixol, gluphentazine decanoate, zuclopenthixol, haloperidol, thiondazine, thiotthixene and trofluperazine.

As used herein, “low dose” of medication means less than a CPZEK of less than about 7. For example, a low dose of risperidone is less than about 5 mg/day for an adult patient.

As used herein, “low D2 dopamine receptor binding” or “low binding” antipsychotics means having an affinity for the D2 dopamine receptor exhibiting a Kᵢ of greater than 15 nM, as measured by in vitro radioligand binding (See, e.g., Levant, 1997, Pharmacological Reviews, 49(3):231-252). This class of antipsychotic medications is often referred to in the art as “atypical” antipsychotics. Representative examples include Olanzapine and Clozapine.

As used herein, “a” or “an” means at least one, unless clearly indicated otherwise.

Methods of the Invention

The invention provides methods of identifying optimal treatment protocols for candidate psychiatric patients or patients with movement disorder for treatment with medication that acts at the D2 dopamine receptor. The method comprises determining a patient's D2 dopamine receptor DRD2) genotype. The determination of whether the patient's DRD2 genotype is Taq1A allele positive (A1+) or Taq1A allele negative (A1−) provides a basis for selecting treatment.

Patients to be treated include those with schizophrenia, post-traumatic stress disorder (PTSD), depression, social anxiety or mixed anxiety and depressive states. In another embodiment, the patient suffers from a movement disorder, such as Parkinson's Disease.

It is accepted psychiatric practice to treat a schizophrenic or other psychotic patient with either a high binding antipsychotic, examples of which include risperidone, flupenthixol, fluphenazine decanoate, zuclopenthixol, haloperidol, thiondazine, thiothixene or trofluperazine, or a low binding atypical antipsychotic, such as olanzapine or clozapine. Patients suffering from PTSD, depression, social anxiety or mixed anxiety and depressive states are typically treated with an SSRI, such as paroxetine or halopram. The methods of the invention can be used by the clinician to guide the selection of an effective medication and dose for individual patients.

Genotyping

Genotyping is typically performed using a blood sample drawn from the subject to be treated. DNA can be extracted from the patient sample using standard techniques and subsequently used as a template for determination of Taq1A DRD2 alleles by polymerase chain reaction (Grandy et al 1993). As described in Noble et al. 1994, the amplification of DNA can be carried out using a Perkin Elmer Gene Amp 9600 thermocycler. Approximately 500 ng of amplified DNA is then digested with 5 units of Taq 1 restriction enzyme (GIBCO/BRL, Grand Island, N.Y.) at 65 degrees centigrade overnight. The resulting products can be analysed by electrophoresis in a 2.5% agarose gel containing ethidium bromide and visualised under ultraviolet light. The A1/A2 genotype is revealed by three fragments: 310 bp, 180 bp, and 130 bp. A2/A2 genotype is indicated by two fragments: 180 bp and 130 bp. The A1/A1 genotype is shown by the uncleaved 310 bp fragment.

Dosage

Those skilled in the art are familiar with appropriate dosage for patients being treated with medications that act at the D2 dopamine receptor, including what is meant by “high dose” and “low dose” antipsychotics. Typically, a low dose treatment with risperidone, for example, would be in the range of 2-4 mg/day for an adult human patient. A high dose would be 6 mg/day or more.

As a guide, one can compare differing medications to the chlorpromazine equivalent per kilogram of body weight (CPZEK). For example, one mg risperidone is equipotent to 100 mg chlorpromazine, 100 mg thioridazine, or 2 mg haloperidol.

While clinicians skilled in the treatment of psychiatric and movement disorders are familiar with high and low dose treatment regimens, the identification of which patients will respond best to high or low dose treatment has previously been a challenge. The methods of the invention can be used in the selection between high dose and low dose therapies, between typical and atypical antipsychotics, and whether to prescribe SSRIs.

Patients having the Taq1A (A1) allele (A1+ allelic status) are candidates for treatment with high dose of high D2 dopamine receptor binding antipsychotics and/or SSRIs that influence D2 dopamine receptor density. A1+ allelic status includes those exhibiting either the A1/A1 genotype or the A1/A2 genotype. Patients lacking the Taq1A allele (A1− allelic status, or A2/A2) are not likely to respond well to these SSRIs, and are candidates for treatment with low dose of low D2 dopamine receptor binding or low dose high D2 dopamine receptor binding atypical antipsychotics.

### EXAMPLES

The following examples are presented to illustrate the present invention and to assist one of ordinary skill in making and using the same. The examples are not intended in any way to otherwise limit the scope of the invention.

### Example 1

### Dose Related Extrapyramidal Adverse Effects in Schizophrenic Patients Treated with Risperidone are Associated with the D2 Dopamine Receptor Gene

The effective treatment of schizophrenia with antipsychotic medication is frequently compromised by adverse effects. Medication induced Extrapyramidal effects (UPS) are associated with decreased compliance and poorer treatment outcome (Gerlach, 2002). Additionally EPS are associated with worsened negative symptoms, dysphoria, impaired cognition and an increased risk of tardive dyskinesia (Tandon, 2002). Anticholinergic medication can be efficacious in ameliorating EPS however these compounds are associated with their own adverse effects such as sexual dysfunction, constipation, dry mouth, urinary retention, impaired cognition and an exacerbation of psychosis (Kopala, 1997). It remains an important clinical challenge to identify medications and dose regimes with strong clinical efficacy and tolerable adverse effect profiles.

Risperidone, a second generation atypical antipsychotic medication, is associated with a lower occurrence of EPS (Love & Nelson, 2000; Heck et al 2000) and tardive dyskinesia (Jeste, et. al. 1999) when compared with conventional, typical antipsychotics. While this has been an important treatment advance EPS are more frequently associated with this medication as dose increases (Sussman, 2002, Love & Nelson, 2000) as a result of increased D2dopamine receptor occupancy (Nyberg et al, 1999). Consequently, the recommended dose of Risperidone has been reduced from 6 mg/day to 4 mg/day, although some patients still experience EPS at this lower dose (Nyberg et al 1999).

The dose of Risperidone that induces EPS varies considerably among patients suggesting differing individual susceptibility. (Nyberg et al, 1999) Individual, physiological factors that influence the development of EPS have not been identified. One such factor may be the Taq1A restriction fragment length polymorphism located in the untranslated region of the D2 dopamine receptor DRD2 gene (Grevle et al, 2000). The A1 (variant) allele (A1/A1 and A1/A2 genotypes) is more commonly found in patients with Parkinson's Disease than in matched controls (Grevle et al 2000) and is also associated with motor fluctuation in affected individuals (Wang et al 2002). Furthermore, this polymorphism is associated with a raised prolactin response to antipsychotic medication (Mihara, 2000, 2001; Young et al in press) reflecting greater antipsychotic D2 occupancy (Markianos et al 2001). As the Taq 1A polymorphism of the DRD2 is associated with both Parkinson's disease and increased antipsychotic D2dopanine occupancy we investigated whether or not patients treated with Risperidone showed differing dose susceptibility for EPS.

Method

SUBJECTS. Forty-seven Caucasian patients attending three psychiatric units for the treatment of their schizophrenia consented for the study. All patients met Diagnostic and Statistical Manual of Mental Disorders (DSM-1V) criteria for schizophrenia. They underwent a clinical history taking by either a psychiatrist (SB, BL, MB, WW) or a clinical psychologist (R.McDY). All participants also underwent a neurological examination and demographic details including ethnic background data was obtained. All subjects had received Risperidone for at least one month at a stable dose. The current dose varied between 2 mg and 6 mg Risperidone per day, 2 mg (N=9), 2.5 mg (N=1), 3 mg (N=13), 4 mg, (N=16), 4.5 mg (N=1), 5 mg (N=2), and 6 mg (N=5). There were no significant differences in dose between A1+ and A1− subjects. Adherence in inpatients was sound as all inpatients were administered medication by nursing staff. Outpatient adherence was estimated by self-report and assessment by their treating psychiatrists.

As other psychoactive agents can influence dopaminergic activity (Jarvinen et al., 1992, Hugues et al 2000, Keltner et al 2002, Basturk et al 2001) those patients on regular antidepressant, opiate, anxiolytic or mood stabilising medication were excluded from the study. All patients were physically well. The average age of those recruited was 33.8 years (sd=12.2 years), and there were 41 males and 8 females. Of the sample, 18 were inpatients at the time of data collection and 31 were outpatients. In terms of clinical and psychosocial history 12 reported a past history of suicide attempts and 13 had a past history of criminal conviction.

PROCEDURE Patients were recruited from the Fortitude Valley Community Mental Health Centre, the Royal Brisbane Mental Health Unit and the Park Psychiatric Hospital. These facilities are located in Brisbane, Australia. Inclusion criteria included being aged between 18 and 65 years, having a DSM IV diagnosis of Schizophrenia and also being cognitively able to participate in order to maximise the validity of data obtained. All subjects possessed an adequate comprehension of English. Potential participants were excluded if they had Schizoaffective Disorder or any other major comorbid psychiatric disorder such as Bipolar Disorder, Dementia, Organic Brain Syndrome, and Major Depressive Disorder with Delusions. EPS were assessed using the scales “Parkinsonism, Dystonia and Dyskinesia: Questionnaire and Behavioral scale (Physician Or Nurse) and “Parkinsonism: Physician's Examination” from the Extrapyramidal Rating Scale (ESRS), (Chounaird, et al, 1980) the most comprehensive rating scale for EPS (De Deyn & Wirshing, 2001). The Questionnaire and Behavioral scale involves a Physician inquiring about, and then rating, symptoms of Parkinsonism, dystonia and dyskinesia. The Physician's examination involves rating expressive automatic movements, bradykinesia, rigidity, gait and posture, tremor, akathisia, sialorrhea, and postural stability.

A 10 mL blood sample was drawn from each subject for DNA extraction and prolactin determination. DNA was sent to the University of California, Los Angeles, for genotyping. All participants provided informed consent and were able to terminate participation at any time without prejudice. Institutional ethics approval was obtained from the clinics and hospitals involved. Demographic and clinical data were combined with genetic data at the completion of the trial.

GENOTYPING A ten ml blood sample was drawn from each subject DNA was extracted using standard techniques and subsequently used as a template for determination of Taq1A DRD2 alleles by polymerase chain reaction (Grandy et al 1993). As previously described (Noble et al 1994) the amplification of DNA was carried out using a Perkin Elmer Gene Amp 9600 thermocycler. Approximately 500 ng of amplified DNA was then digested with 5 units of Taq 1 restriction enzyme (GIBCO/BRL, Grand Island, N.Y.) at 65 degrees centigrade overnight. The resulting products were analysed by electrophoresis in a 2.5% agarose gel containing ethidium bromide and visualised under ultraviolet light. The A1/A2 genotype is revealed by three fragments: 310 bp, 180 bp, and 130 bp. A2/A2 genotype is indicated by two fragments: 180 bp and 130 bp. The A1/A1 genotype is shown by the uncleaved 310 bp fragment.

Data Analysis

Information coded from interview proformas was entered into a computer data base. Doses of Risperidone were converted to chlorpromazine equivalents per kilo of body weight. A median split was performed in order to separate individuals receiving median dose or above (high dose, n=24) from those receiving below median dose flow dose, n=23) Subjects were then divided into four groups; A1 subjects receiving below the median dose risperidone, (A1 low dose); A1 subjects receiving the median dose or above of risperidone, (A1 high dose), A2 subjects treated with below the median dose, (A2 low dose); A2 subjects treated with the median dose or above, (A2 high dose). Univariate analysis of variance (ANOVA) was used to test between-subject effects. A p-value of <0.05 was considered to be statistically significant.

Results

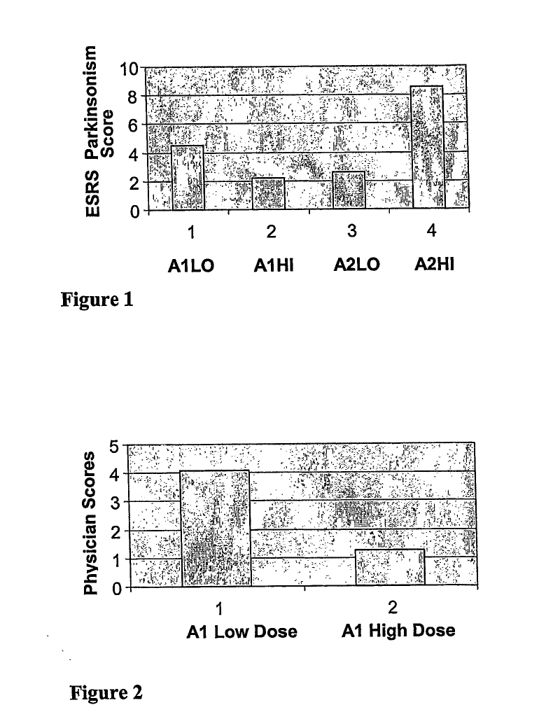

The 47 subjects had the following genotypes: A1A1 (A1 allele) n=3, A1A2 (A1 allele), n=23; and A2A2 (A2 allele), n=21. There was no significant difference in the ages of the high and low dose groups (df1,45; F=2.14,p=0.150) and A1+ and A1− allelic subjects. FIG. 1 presents mean ESRS Parkinsonism Physician's Examination scores of the four groups; A1 low dose (n=15); A1high dose, (n=11); A2 low dose, (n=8); A2 high dose, (n=13). The means and standard deviations of these ESRS scores in particular groups were: A1 low dose, mean=4.53, S.D.=4.357; A1 high dose, mean=2.18, S.D.=2.56; A2 low dose, mean=2.63, S.D.=3.29; A2 high dose, mean=8.54, S.D.=6.00. There was a significant gene by dose interaction (df 1,43, F=5.189, p=0.028). At high doses of Risperidone A2 subjects were rated by physicians as having significantly worse symptoms of Parkinsonism than A1 subjects.(df 1,23, F=4.790, p=0.039) At low doses there was a trend for A1 subjects having more severe symptoms of Parkinsonism than A2 subjects.(df 1,21, F=3.4, p=0.08)

Discussion

The essential finding of this Example is that A1 and A2 individuals exhibit different dose susceptibility for EPS when treated with Rispetidone. This may be due to the marked differences in D2 physiology between these groups.

There is compelling evidence that A1+ individuals have a reduced density of brain D2 dopamine receptors (Noble et al, 1991, Thompson et al, 1997, Pohjalainen et al, 1998, Jonsson, et al, 1999). An early brain autopsy study (Noble et al., 1991) found a significant reduction of approximately 30% in the number of D2 dopamine receptors (Bmax) in the caudate nucleus of A1+ compared to A1− allelic subjects. Moreover, a significant progressive decline in Bmax was found across A2/A2, A1/A2, A1/A1 genotypes in that order. There was no difference in D2 dopamine binding affinity (kd between A1+ and A1− allelic subjects. Thompson et al., 1997 also reported 30%-40% reduction in D2 dopamine receptor density in the striatum of A1+ individuals compared to A1− individuals. An in-vivo study of healthy Finnish volunteers (Pohjalainen et al., 1998) showed significantly decreased D2 receptor density in the striatum of A1+ when compared with A1− allelic subjects. Again, there was no difference in the kd between the two groups. In Jonsson et al (1999), another PET study of healthy humans using (11C) raclopride, a significant association of the A1 allele with low D2 dopamine receptor density was found. While, Laruelle et al (1998) reported no association between reduced D2 binding and the A1 allele subjects an editorial (Hitzemann, 1998) suggested the study had insufficient power to detect a significant difference between A1+ and A1− individuals.

The most likely explanation of the association of the A1 allele, a polymorphism in a non-coding region of the DRD2 gene, with reduced D2 receptor density, is that the sequence variation causing the Taq1 A polymorphism is in linkage disequilibrium with functional allelic variants that affect receptor expression (Noble, et al, 1998, Comings et al, 1991, O'Hara et al 1993, Arinami et al, 1997). The Taq 1A variants are now known to be in linkage disequilibrium with C957T, a synonymous mutation in the human DRD2. Furthermore, C957T affects mRNA folding leading to both less stable mRNA and decreased translation. These effects dramatically diminish dopamine induced up-regulation of D2 receptors (Duan et al, 2003).

A1+ individuals with genetically determined reduced D2 density have D2 receptors that are functionally identical to those found in A1− individuals. The binding affinity for dopamine (Kd) is identical in both A1+ and A1− individuals.(Noble et al 1991, Pohjalainen 1998) A1+ individuals will, therefore, at any given dose of antipsychotic medication, tend to have a lower density of free, unbound, D2 receptors and consequently, greater drug D2 occupancy. Additionally A1 individuals have a diminished ability to upregulate D2 receptors in response to blockade (Duan et al, 2003). A1 individuals have significantly worse Parkinsonian symptoms at doses below the median than at doses above the median. Furthermore A2 subjects treated with above median doses of Risperidone are rated as having worse EPSES than A1 subjects treated with the same dose of medication. The susceptibility of A1 subjects at low doses of Risperidone probably reflects the lower density of free, unbound D2 receptors found in this group. Higher doses of Risperidone are required to induce EPSES in A2 individuals due to their greater D2 density and greater capacity for D2 upregulation. At high doses of Risperidone, A1 subjects experience minimal EPSES. Why this occurs is not known however high doses (>100 mg per day) of haloperidol employed in the 1970s for treatment resistant schizophrenia were not associated with serious extrapyrnmidal side effects. (McCreadie R g and MacDonald I M, 1977) Lower density of D2 receptors found in A1 subjects combined with a higher dose of the “tight binding” agent Risperidone (David et al 2000) is likely to result in very few unbound D2 receptors in this group of patients. Perhaps very high D2 drug occupancy results in a suppression of EPSES.

This Example provides an association study of a clinical population. These observations are likely to be of significant practical assistance to treating clinicians in allowing prediction of patients who are likely to experience Parkinsonian symptoms at low antipsychotic dose and will provide a rational strategy for consequent drug dosing. For example, this may indicate that in A1− patients with EPS the dose should not be increased but in A1+ patients an increased dose may ameliorate the symptoms. This may also result in an improved antipsychotic effect in A1+ patients.

### Example 2

### Antipsychotic-Related Prolactin Elevation is Found in Schizophrenics Carrying the DRD2 A1 Allele

Hyperprolactinemia induced by antipsychotic medication may be involved in the development of a variety of serious health problems including breast cancer, osteoporosis, cardiovascular disorders and sexual dysfunction. In a sample of 144 schizophrenic patients treated with commonly used antipsychotic medication there was a linear increase in prolactin levels across drug groups reflecting markedly different drug D2 binding affinities. Furthermore, patients with A1+ DRD2 gene status (A1/A1, A1/A2 genotypes) had significantly higher serum prolactin than A1− individuals (A2/A2 genotype) (p<0.036) reflecting greater functional D2 drug occupancy in the A1+ group. Among individuals treated with Clozapine those with the A1+ allele had significantly higher prolactin than their A1− counterparts (p=0.0398). DRD2 variants may be associated with different antipsychotic treatment responses and liability for adverse drug effects. Pharmacogenetic implications of these findings are presented.

Introduction

Hyperprolactinemia produced by antipsychotic medication is the result of drug D2 receptor occupancy and consequent blockade of the inhibitory effects of dopamine. (Markianos et. al. 2001). Dopamine is released by the hypothalamus into the hypophyseal portal system and is delivered to the anterior lobe of the pituitary. Dopamine subsequently inhibits prolactin release by binding to D2 receptors. Bound receptors induce changes in membrane channels and G proteins thus suppressing lactotroph prolactin secreting activity. Furthermore, dopamine activates intracellular signalling pathways which decrease gene expression and result in reduced lactotroph proliferation (Iaccarino et. al. 2002). Dopamine release in the hypothalamus is regulated by a variety of mechanisms including oestrogen, thyroid releasing factor, endogenous opioids, psychological and physical stress, neuropeptides, illness (for example, epilepsy and herpes zoster infection), neurotransmitter activity and prolactin itself (Ben-Jonathan & Hnasko, 2001, Petty, 1999).

Hyperprolactinemia has been considered an inevitable consequence of treatment with any typical antipsychotic agent (Petty, 1999). It may result in depression, sexual dysfunction, amennhorea, galactorrhea, breast cancer and osteoporosis (Halbreich et al 2003). Depression and sexual dysfunction induced by hyperprolactinemia have the potential to adversely influence adherence to treatment (Maguire, 2002) and additional health problems can exacerbate the burden experienced by those with schizophrenia.

Antipsychotics vary widely in their binding affinity for the D2 receptor. Several currently available compounds, such as Clozapine and Quetiapine have a lower D2 binding affinity than dopanine (Remington & Kapur 2000) and have not been associated with hyperprolactinemia (Markianos et al 2002). By contrast, hyperprolactinemia is mote commonly associated with tighter binding agents such as risperidone and typical antipsychotics (David et al, 2000). Olanzapine, an atypical antipsychotic with intermediate D2 binding affinity has generally been associated with modest increases in prolactin levels (David et al, 2000) and the prolactin levels observed are not an accurate reflection of drug D2 occupancy (Lavalaye et al 1999). In support of this prolactin levels are elevated in patients treated with risperidone, but not when treated with olanzapine, despite comparable D2 drug occupancy levels measured by [123I ] iodobenzamide SPECT (Lavalaye et al, 1999).

Although use of tighter binding agents is generally associated with higher prolactin levels it is a common clinical observation that there are considerable individual variations in prolactin levels induced by identical medication at a given dose. Individual vulnerability factors that are associated with the development of hypetprolactinemia are likely to involve D2 receptor physiology given the central role of these receptors in prolactin secretion regulation. The A1 allele of the D2 dopamine receptor gene (DRD2) (A1/A1 and A1/A2 genotypes) is associated with significantly reduced density of D2 receptors (Noble et al, 1991). Nemonapride, a selective D2 antagonist, is associated with significantly elevated prolactin in A1+ females (A1/A1 and A1/A2 genotypes) with schizophrenia (Mihara et al 2000). A1+ allelic status is also associated with increased prolactin response to bromperidol in female inpatients with schizophrenia (Mihara et al 2001). In the current study we investigated the possible association of A1+ allelic status with raised prolactin levels in both male and female schizophrenic patients treated with antipsychotics frequently used in clinical practice. The current study investigated the prolactin response to a variety of agents classified as tight (Risperidone, Typicals) intermediate (Olanzapine) or loose (Clozapine) binding (Remington & Kapur, 2000).

Method

SUBJECTS. One hundred and forty four unrelated Caucasian patients (123 males, 21 females) attending various psychiatric units for the treatment of their schizophrenia were recruited for the study. Nine of the women were post menopausal. All patients met Diagnostic and Statistical Manual of Mental Disorders (DSM-1V) criteria for schizophrenia. They underwent a clinical history taking by either a psychiatrist (SB, BL, MB, WW) or a clinical psychologist (R. McDY). Demographic details including ethnic background data was obtained.

All subjects had received the same antipsychotic medication for at least one month at a stable dose. As other psychoactive agents can influence prolactin levels (Jarvinen et al., 1992, Hugues et al 2000, Keltner et al 2002, Basturk et al 2001) those patients on regular antidepressant, opiate, anxiolytic or mood stabilising medication were excluded from the study. Women on the oral contraceptive pill were excluded. No patients had received prn benzodiazepines for at least 24 hours. Inpatients had not received more than 5 mg of prn diazepam equivalent in any 24 hour period during their admission. All patients were physically well and none had a history of epilepsy. The average age of those recruited was 36.41±12.01 years. At the time of data collection, 61 subjects were inpatients and 83 were outpatients. The breakdown of medications prescribed was as follows: 31 patients were on Clozapine, 31 on Olanzapine, 33 on Typicals (this included 12 patients on Flupenthixol, 2 on Fluphenazine decanoate, 13 on Zuclopenthixol, 3 on Haldon, 1 on Thioridazine, 1 on Thiothixene and 1 on Trufluperazine) and 49 on Risperidone. The dose of the antipsychotics was transformed to Chlorpromazine equivalents per kilograms (CPZEK). The mean mg. CPZEK dose±SD of each of the four drug groups used was: Clozapine, 5.14±2.94; Olanzapine, 5.37±2.62; Typicals, 5.60±3.70; Risperidone, 4.82±2.00. There were no significant differences in dose used among the four drug groups (F (3,128)=0.51, P=0.67). Weight data was missing on 12 subjects (1 Clozapine patient, 2 Olanzapine patients, 2 Risperidone patients and 7 Typicals patients).

Adherence in inpatients was complete as all subjects were administered medication by nursing staff. Outpatient adherence was estimated by self-report and assessment by the treating psychiatrist. Thirty of the 33 patients receiving typical medication were treated with nurse administered depot preparations.

PROCEDURE Patients were recruited from the Fortitude Valley Community Mental Health Centre, the Royal Brisbane Mental Health Unit and the Park Psychiatric Hospital. These facilities are located in Brisbane, Australia. Inclusion criteria included being aged between 18 and 65 years and having a DSM IV diagnosis of Schizophrenia. As noted all subjects were prescribed the same antipsychotic for at least a month on a stable dose (for at least 2 weeks). Patients taking any regular additional antidepressant, anxiolytic or mood stabilising psychotropic medication were excluded. All subjects had to possess an adequate comprehension of English. Potential participants were excluded if they had any other psychiatric disorder including Schizoaffective Disorder, Bipolar Disorder, Dementia, Organic Brain Syndrome, Major Depressive Disorder with Delusions.

A 10 mL blood sample was drawn from each subject for DNA extraction and prolactin determination. DNA was sent to the U.C.L.A. for genotyping. All participants provided informed consent and were able to terminate participation at any time without prejudice. Institutional ethics approval was obtained from the clinics and hospitals involved. Prolactin determination using mIU/(milli international units per litre) was conducted at the Royal Brisbane Hospital. Prolactin and demographic data were combined with genetic data at the completion of the trial.

GENOTYPING. DNA was extracted from leucocytes using standard techniques and subsequently used as a template for determination of Taq1A DRD2 alleles by polymerase chain reaction (Grandy et al 1993) As previously described (Noble et al 1994) the amplification of DNA was carried out using a Perkin Elmer Gene Amp 9600 thermocycler. Approximately 500 ng of amplified DNA was then digested with 5 units of Taq 1 restriction enzyme (GIBCO/BRL, Grand Island, N.Y.) at 65 degrees centigrade overnight. The resulting products were analysed by electrophoresis in a 2.5% agarose gel containing ethidium bromide and visualised under ultraviolet light. The A1/A2 genotype is revealed by three fragments: 310 bp, 180 bp, and 130 bp. A2/A2 genotype is indicated by two fragments: 180 bp and 130 bp. The A1/A1 genotype is shown by the uncleaved 310 bp fragment. Subjects with the A1/A1 and A1/A2 genotypes were considered to have the A1+ allelic status, while those with the A2/A2 genotype were considered to have the A1− allelic status.

Data Analysis

Information coded from interview proformas was entered into a computer data base. Chi-square test (Yates corrected) and Fisher's exact test, where appropriate, were employed to compare differences in non continuous variables between A1+ and A1− allelic groups. Analysis of variance (ANOVA) was used to compare differences among the various drug groups in prolactin levels as a continuous variable. Similarly, one-way ANOVA was employed to examine differences in prolactin levels between A1+ and A1− allelic groups. A p-value of <0.05 was considered to be statistically significant.

Results

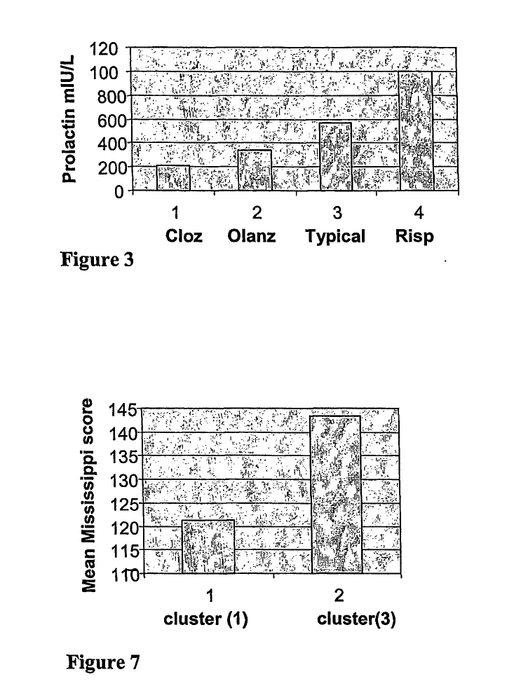

Consistent with other studies of patients on antipsychotics there was a significant effect for gender with females obtaining significantly higher prolactin elevation than males F (1,142)=25.19, p<0.0001). Males and females were equally represented across the four drug groups. There was also a significant difference in prolactin response among the four antipsychotic treatment groups F (3,140)=19.009, p<0.0001. FIG. 3 shows prolactin levels in each of the treatment groups. Olanzapine compared to Clozapine treatment resulted in significantly higher prolactin levels (F (1, 60)=4.76, P=0.033). Patients treated with Typical antipsychotics had significantly elevated prolactin levels than their Olanzapine treated counterparts (F(1,62)=7.60, P=0.007. Finally, significantly higher levels of prolactin occurred in patients treated with Risperidone compared to those treated with Typical antipsychotics (F(1,80)=8.97, P=0.004).

The genotypes of the 144 patients were as follows, A1/A1 (n=7), A1/A2 (n=55.2) and A2/A2 (n=82). The age of the A1+ allelic group (35.5±12.7) was not significantly different from that of the A1− allelic group (37.1±11.4) (F(1,141)=0.66, P=0.42). There were no significant differences in gender (χ2 (1)=0.048, p=0.83), inpatient or outpatient status (χ2 (1)=0.00, p>0.99), family history of schizophrenia (X2(1)=0.00, p>0.99), criminality (χ2(1)=0.02, p=0.90), binge drinking (χ2(1)=0.08, p=0.78) or suicide attempts (χ2(1)=2.026, p=0.16) between A1+ and A1− allelic subjects. There was also no difference in antipsychotic dose between A1+ and A1− allelic groups taking Clozapine (F(1,28)=2.36, P=0.14), Olanzapine (F(1,27)=1.0., P=0.33), Typicals (F(1,24)=0.033, P=0.69), and Risperidone (F(1,45)=0.70, P=0.50). In terms of clinical status there were no differences in umber of admissions F(1,140)=1.447, p=0.230, PANSS positive symptoms F(1,140)=0.60, p=0.8061, PANSS Negative symptoms F (1, 139)=2.604, p=0.1089).

Table 1 shows serum prolactin levels of A1+ and A1− allelic schizophrenic patients treated with antipsychotic drugs. Using analysis of variance, when all the antipsychotics were considered together patients carrying the A1+ allele had a significant and about a 40% higher Prolactin levels than patients carrying the A1− allele (F(1, 142)=4.50, P=0.036). When individual drug groups were examined, only the loosest binding antipsychotic Clozapine showed a significant and a more than two fold higher Prolactin levels in A1+ compared to A1− allelic patients (F(1,29)=4.63, P=0.04). To test whether this result was influenced by gender the analysis was rerun for males only as there were insufficient females to analyse and significance remained F(1,26)=4.58, p=0.042). In none of the other three groups of antipsychotics were there significant differences in Prolactin levels between A1+ and A1− allelic patients.

Hyperprolactinemia was defined using community sample cut-off levels set at a 95% reference range, 430 mU/I in men and 560 mU/l in women (Vanderpump, French, Appleton, Tunbridge & Taylor, 1998). In total 64 patients, or 44% of the sample, exceeded these levels. Only 7 of these patients were prescribed the low binding agents, Clozapine and Olanzapine confirming that these medications are rarely associated with hyperprolactinemia (5% of the sample). Disturbingly 40 of the patients on Risperidone exceeded these prolactin levels, indicating that 81% of patients on this medication were in the hyperprolactinemic range. A Yates corrected chi-square analysis conducted to compare allelic status across the groups with prolactin levels in the normal range and those with hyperprolactinemia was significant (χ2(1)=5.523 , p=0.018).

TABLE 1
Serum prolactin (in IU/l) means of A1+ and A1− allelic
schizophrenics treated with various antipsychotics

| Group | A1+ Allele | A1− Allele |
|---|---|---|
| All Subjects, N = 144 | 708 ± 79, n = 62 | 499 ± 62, n = 82 |
| Clozapine, N = 31 | 310 ± 92, n = 12 | 147 ± 18, n = 19 |
| Olanzapine, N = 31 | 327 ± 36, n = 9 | 346 ± 63, n = 22 |
| Typical Antipsychotics, | 622 ± 94, n = 14 | 531 ± 97, n = 19 |
| N = 33.3 | | |
| Risperidone, N = 49 | 1058 ± 142, n = 27 | 928 ± 171, n = 22 |

Discussion

Prolactin response to antipsychotic medication was greatest in risperidone followed by typicals, olanzapine and clozapine and was greater in females than males. This increasing order across medications is also found in positron emission tomography (PET) binding studies and confirms identical results described in a previous study (Markianos et al 2001). In that study prolactin response to antipsychotic medication reflected antipsychotic D2 receptor occupancy.

Overall, A1+ individuals have significantly higher prolactin levels when treated with a variety of antipsychotic medications. Furthermore, A1+ schizophrenics have significantly higher prolactin levels than A1− patients when treated with the loose binding agent clozapine. The greater prolactin response to antipsychotics witnessed in A1+ schizophrenics in this, and other, (Mihara 2000, 2001) studies indicates that A1+ individuals treated with antipsychotic medication generally have greater functional drug D2 occupancy, that is that they have fewer unbound dopanine receptors. Drug D2 binding and individual DRD2 variants are both important factors in determining individual prolactin response to antipsychotic medication. Despite the elevation taking clozapine the majority of the cases with hyperprolactinemia were being administered tight binding agents, there was also a strong allelic effect with A1+ individuals being significantly more likely to be classified as hyperprolactinemic than their A1− counterparts.

There is compelling evidence that A1+ individuals have a reduced density of brain D2 dopamine receptors (Noble et al, 1991, Thompson et al, 1997, Pohjalainen et al, 1998, Jonsson, et al, 1999). An early brain autopsy study (Noble et al., 1991) found a significant reduction of approximately 30% in the number of D2 dopamine receptors (Bmax) in the caudate nucleus of A1+ compared to A1− allelic subjects. Moreover, a significant progressive decline in Bmax was found across A2/A2, A1/A2, A1/A1 genotypes in that order. There was no difference in D2 dopaine binding affinity (1(d) between A1+ and A1− allelic subjects. Thompson et al., 1997 also reported 30%-40% reduction in D2 dopamine receptor density in the striatum of A1+ individuals compared to A1− individuals. An in-vivo study of healthy Finnish volunteers (Pohjalainen et al., 1998) showed significantly decreased D2 receptor density in the striatum of A1+ when compared with A1− allelic subjects. Again, there was no difference in the Kd between the two groups. In Jonsson et al (1999), another PET study of healthy humans using (11C) raclopride, a significant association of the A1 allele with low D2 dopamine receptor density was found. While, Laruelle et al (1998) reported no association between reduced D2 binding and the A1 allele subjects an editorial (Hitzemann, 1998) suggested the study had insufficient power to detect a significant difference between A1+ and A1− individuals.

The most likely explanation of the association of the A1 allele, a polymorphism in a non-coding region of the DRD2 gene, with reduced D2 receptor density, is that the sequence variation causing the Taq1 A polymorphism is in linkage disequilibrium with functional allelic variants that affect receptor expression Noble, et al, 1998, Comings et al, 1991, O'Hara et al 1993, Arinami et al, 1997). The Taq 1A variants are now known to be in linkage disequilibrium with C957T, a synonymous mutation in the human DRD2. Furthermore, C957T affects mRNA folding leading to both less stable mRNA and decreased translation. These effects dramatically diminish dopamine induced up-regulation of D2 receptors (Duan et al, 2003).

A1+ individuals with genetically determined reduced D2 density have D2 receptors that are functionally identical to those found in A1− individuals. The binding affinity for dopamine (Kd) is identical in both A1+ and A1− individuals.(Noble et al 1991, Pohjalainen 1998) A1+ individuals will, therefore, at any given dose of antipsychotic medication, tend to have a lower density of free, unbound, D2 receptors and consequently, greater drug D2 occupancy. This density reflects an active, dynamic, process involving drug displacing dopamine from the receptor and vice versa.

D2 receptor-drug occupancy and consequent D2 receptor blockade is necessary for both clinical antipsychotic action (Kaput & Remington, 2001a) and a variety of other effects. Studies with conventional antipsychotics report that approximately 70% occupancy results in maximal therapeutic efficacy Nordstrom et al 1993). A trend towards improved efficacy in treatment resistant patients has been found when doses of olanzapine were increased to an average of 30.4 mg (Volavka et al 2002). Preliminary investigations have been undertaken to increase the effectiveness of clozapine, an agent with a high D2 dissociation constant, by adding haloperidol, an agent with a low dissociation constant. (Kapur et al 2001b) A1− individuals are more likely to benefit from these approaches to improve drug D2 receptor occupancy as they have more free, unbound, D2 receptors at any given dose. Conversely A1+ individuals may not derive as much improvement as they have higher functional occupancy. Optimal therapeutic effect is likely to be obtained at lower doses in A1+ schizophrenics A1− patients may require a higher dose for maximal antipsychotic effect, particularly when prescribed a loose binding antipsychotic such as clozapine or quetiapine. According to the rapid dissociation model (Kapur & Seeman, 2002) atypical agents hypothesised to have an antipsychotic action without causing other effects such as raised prolactin or extrapyramidal side effects (Kapur & Seeman, 2001). Our data are not consistent with this because dozapine has definite effects on serum prolactin levels with A1+ individuals having significant raised prolactin levels compared with A1− patients. D2 blockade effect of clozapine in A1+ patients is not limited to an antipsychotic effect alone. For example, D2 receptor occupancy also correlates with liability to extrapyramidal adverse effects in patients treated with risperidone (Yamada et al 2002) and a variety of antipsychotics including dozapine (Broich et al 1998), haloperidol (Kapur et al 2000) and olanzapine Gauss et al 1998). A1+ individuals treated with antipsychotic medication are likely to experience extrapyramidal adverse effects at lower dose than A1− patients, as these patients have decreased nigrostriatal D2 receptor density (Thompson et al, 1997).

This Example implicates the DRD2 gene as an important determinant of D2 occupancy in schizophrenic patients treated with antipsychotic medications. A1+ patients generally display higher prolactin levels and hence greater functional drug D2 occupancy than A1− individuals. Our results demonstrate that this association is most evident with the loose D2-binding antipsychotic clozapine. The D2 blocking antipsychotic effect of clozapine may not therefore occur independently of other effects.

### Example 3

### D2 Dopamine Receptor Gene Polymorphism Differentiates Paroxetine Treatment Outcome in Posttraumatic Stress Disorder

To determine whether allelic status of the D2 dopamine receptor (DRD2) gene differentiates response to a selective serotonin reuptake inhibitor in the treatment of posttraumatic stress disorder (PTSD). Sixty-five Caucasian war veterans with combat-related PTSD were treated with paroxetine for 8 weeks. Patients were assessed at baseline and at follow-up using the General Health Questionnaire-28 (GHQ). TaqI A DRD2 alleles were determined by PCR. There was a trend for more DRD2 A1− (A2A2) than A1+ (A1A1) allelic patients to discontinue treatment due to adverse events. In the remaining 45 patients, significant improvement (P=0.015) in the GHQ total score occurred over the treatment period. However, when patients were classified by their allelic status, GHQ total score of A1+ patients showed a significant positive treatment response (P=0.010), whereas the A1− patients did not. Similarly, a significant positive outcome was found in three of the four GHQ subscale scores in A1+ compared to A1− patients. These included anxiety/insomnia (P=0.013), social dysfunction (P=0.005), and depression (P=0.016). DRD2 variants predict response to paroxetine in PTSD. The study suggests a pharmacogenetic approach to the treatment of this disorder.

Introduction

Combat-related posttraumatic stress disorder (PTSD) is a highly debilitating condition with a chronic course. The quality of life of PTSD patients is frequently compromised by comorbid conditions such as social anxiety disorder, panic disorder, generalized anxiety disorder, dysthymia and major depressive disorder (Zatzick et al., 1997; O'Toole et al., 1998). Selective serotonin reuptake inhibitors (SSRIs) are an effective treatment for a wide variety of psychiatric disorders. These include PTSD, depression, social phobia, and mixed anxiety and depressive states. Indeed, SSRIs are generally accepted to be the first line pharmacothetapy for PTSD (Hidalgo and Davidson, 2000; Ballinger et al., 2000).

SSRI treatment outcomes are variable. A recent randomized controlled trial of the SSRI sertraline in the treatment of PTSD revealed a responder rate of 53% compared to 32% with placebo (Brady et al., 2000). Another study reported that only 50% of chronic PTSD patients experienced substantial improvement in their symptoms with antidepressant treatment (Dow and Kline, 1997). Studies in social phobia, major depressive disorder and dysthymia indicate similar SSRI response rates of 55% (Stein et al., 1998), 69% (Van Houdenhove et al., 1997) and 59% (Thase et al., 1996) respectively. Individuals across diverse diagnostic groups show similar SSRI response rates of approximately 20% greater than placebo and 30-50% of patients do not respond to SSRI medication. Furthermore, the rates of response to different SSRIs are not significantly different despite their varying pharmacological profiles (Nelson, 1999). Moreover, few patient predictors of response have been identified to assist the clinician in the selection of an antidepressant (Nelson, 1999).

The monoamine deficiency hypothesis (Delgado, 2000) predicts that depression is a consequence of depleted central nervous system monoamine (noradenaline, serotonin and dopamine) levels. This hypothesis is supported by the observation that antidepressants increase the levels of various brain monoamines. However, despite much research attention, a primary dysfunction has not been found in the brain monoamine systems of patients suffering from depressive disorders (Delgado, 2000). Regarding PTSD, dopaminergic tone is diminished in response to stress which results in a hypodopaminergic state amongst sufferers (Deutch and Roth, 1990; Deutch and Young, 1995). SSRIs have a significant impact on dopaminergic function, with stimulation of 5-HT 1A and 5-HT 1B receptors facilitating dopamine release and stimulation of the 5-HT 2 receptor inhibiting dopamine release (Ng et al., 1999; Rollema et al., 2000; Gobert et al., 2000). In vivo brain microdialysis studies indicate that the SSRIs fluoxetine and fluvoxamine increase dopamine efflux (Mendlin et al., 1998).

The mesolimbic dopamine system has been implicated in rapid antidepressant action (Wilner, 1997). D2 dopamine receptors, are particularly important in this antidepressant therapeutic action. Interestingly, paroxetine's antidepressant response is greater in subjects with low D2 dopamine responsivity (Healey and McKeon, 2000). Moreover, striatal D2 dopamine receptor binding, measured by (¹²³I) IBZM, using single photon emission computed tomography, is lower at baseline in SSRI treatment responders than in depressed non-responders and non-depressed controls (Klimke et al., 1999). Furthermore, increases in D2 dopamine receptor binding over time were found in treatment responders, and conversely, treatment non-responders showed decreases in D2 dopamine receptor binding (Klimke et al., 1999).

Given the association of brain D2 dopamine receptor levels and responsivity to SSRIs, an investigation of variants of the D2 dopamine receptor (DRD2) gene on SSRI treatment outcome is warranted. An in vitro study using (³H)spiperone (Noble et al., 1991) as a D2 dopamine receptor ligand, found a significant decrease in the number of D2 dopamine receptors in the brains of subjects with the DRD2 A1+ (A1A1 and A1A2 genotypes) allele than in those with the A1− (A2A2 genotype) allele. An autoradiographic study (Thompson et al., 1997) using (³H)raclopride as a D2 dopamine receptor ligand found significantly reduced D2 dopamine receptor binding in the brains of A1+ compared with A1− allelic subjects. An in vivo positron emission tomography (PET) study, using (¹¹C)raclopride, also found a significant reduction in brain D2 dopamine receptor density in healthy subjects with the A1+ allele (Pohjalainen et al., 1998). Moreover, a more recent PET study of healthy humans (Jönsson et al., 1999), again using (¹¹C)raclopride, found a significant association of the Al allele with measures of low D2 dopamine receptor density. Another study (Laruelle et al., 1998) determined in healthy controls and in schizophrenics D2 dopamine receptor binding potential using (¹²³I) IBZM. No significant difference in this combined sample was found in the D2 dopamine receptor binding potential between A1+ and A1− allelic subjects. However, when the controls and schizophrenics were separately examined, a trend for a lower binding potential was found in A1+ allelic controls, whilst a trend for a higher binding potential was noted in A1+ allelic schizophrenics when compared to their respective A1− allelic subjects. Since two of the above studies (Pohjalainen et al., 1998; Laruelle et al., 1998) appeared in the same journal issue, an editorial (Hitzemann, 1998) reviewed their merits. It suggests that the study using (¹²³I) IBZM (Laruelle et al., 1998) had insufficient power to detect a significant difference between A1+ and A1− allelic controls. Moreover, since schizophrenics showed a trend in the opposite direction, the results on D2 dopamine receptor binding potential and allelic association in these subjects may have been confounded by prior neuroleptic treatment. Indeed, in a recent PET study (Silvestri et al., 2000) using (¹¹C)raclopride, increased D2 dopamine receptor binding was shown in schizophrenic patients subsequent to neuroleptic treatment.

If variants of the DRD2 gene are associated with lower number of D2 dopamine receptors, and if SSRI treatment is more efficacious in subjects with decreased D2 dopamine receptor binding, would variants of the DRD2 gene predict SSRI response? In the present Example, behavioral outcome based on TaqI A DRD2 variants was ascertained in PTSD patients treated with paroxetine.

Materials and Methods

Sixty-five unrelated male Caucasian patients with the diagnosis of PTSD were recruited for study. All subjects were Vietnam combat veterans who had served in the Australian armed forces. None were being treated with psychotropic medication. Patients were excluded from the study if they had a diagnosis of psychosis, bipolar disorder, obsessive compulsive disorder, organic brain syndrome, glaucoma, cardiac disease, or were being treated with anticoagulants or drugs affecting hepatic metabolism (Aropax® [paroxetine] product information, 1999).

Patients were assessed for PTSD by a consultant psychiatrist (BL) or a psychiatric registrar (BK) using Diagnostic and Statistical Manual of Mental Disorders (DSM-IV) criteria. PTSD caseness was confirmed by all patients exceeding the clinical cut-off score of 94 on the Mississippi Scale for combat-related posttraumatic stress disorder (Zatzick et al., 1997). The validity and reliability of the Mississippi scale ate well established in veterans (Keane et al., 1988). Patients then underwent clinical history taking by a psychiatrist or by a clinical nurse (EP). Demographic data and ethnic background were also obtained.

After initial assessment, patients were medically examined and then were started on 20 mg/day of paroxetine for the first two weeks followed by a 40 mg/day dose for the remaining six weeks. It should be noted that response to this drug commences after one week of treatment but does not exceed placebo until the second week of therapy (Aropax® [paroxetine] product information, 1999).

The General Health Questionnaire-28 (GHQ) was administered at baseline (0-7 days on paroxetine) and at the end of treatment (8 weeks after commencement on paroxetine). This questionnaire measures four psychopathological factors particularly relevant to PTSD and its comorbid psychiatric conditions: somatic concerns (GHQ1), anxiety/insomnia (GHQ2), social dysfunction (GHQ3) and depression (GHQ4). The GHQ has been widely validated internationally as a means of detecting psychiatric caseness and is sensitive to change (Ormel et al., 1989). The GHQ has utility as a follow-up measure of veteran mental health following exposure to combat (Deahl et al., 1994) and is sensitive to changes in combat-related PTSD symptoms (Ward, 1997).

A 10 ml blood sample was drawn from each patient. Genomic DNA was extracted employing standard techniques and used as a template for determination of TaqI A DRD2 alleles by the polymerase chain reaction (Grandy et al., 1993). The amplification of DNA was carried out using a Perkin Elmer GeneAmp 9600 thermocycler. Approximately 500 ng of amplified DNA was digested with five units of TaqI restriction enzyme (New England Biolabs) at 65° C. overnight. The resulting products were separated by electrophoresis in a 2.5% agarose gel containing ethidium bromide and visualized under ultraviolet light. Three genotypes are obtained: the A1A2 genotype is revealed by three fragments: 310 bp, 180 bp and 130 bp. The A2A2 genotype by two fragments: 180 bp and 130 bp. The A1A1 genotype is shown by the uncleaved 310 bp fragment. A1+ allelic subjects are those that either have the A1A1 or A1A2 genotype; A1− allelic subjects have the A2A2 genotype only.

Patient assessments were conducted blind to their DRD2 allelic status. In addition to their standard treatment, patients were monitored twice for adverse events. Drug compliance was checked by pill count. All participants provided written informed consent and were able to terminate treatment without prejudice. However, those terminating participation were asked to provide reasons for their withdrawal. Institutional ethics approval was obtained from the Greenslopes Private Hospital.

Information coded from the interviews, GHQ and genotyping results were entered into a computer database. Nominal data were analyzed by Yates corrected X² test and continuous data, with repeated measures, were analyzed by paired t-test or by analysis of variance. P values≦0.05 were considered statistically significant. P values≧0.05 but <0.10 were considered to be approaching a significant level.

Results

The mean age±S.E. of the 65 patients who entered treatment was 51.4±0.7 years. They had the following genotypes: A1A2 (A1+ allele), n=26; and A2A2 (A1− allele), n=39. This genotype distribution did not deviate from Hardy-Weinberg equilibrium (χ²=2.86, P=0.091). There was no significant difference in the ages of A1+ (51.1±0.7 years) and A1− (51.6±1.0 years) allelic subjects (F(1,63)=0.13, P=0.72).

Of the 65 patients who entered the study, 20 (4 A1+ and 16 A1−) discontinued paroxetine treatment for a variety of reasons. A trend (P=0.055) was found in the greater number of A1− than A1+ allelic subjects who discontinued treatment In the group that discontinued treatment, 2 A1+ and 11 A1− allelic subjects had adverse events relating to anxiety, insomnia, headache or tremor. In addition, 2 A1+ and 5 A1− allelic subjects experienced erectile dysfunction, decreased libido or delayed ejaculation.

The 45 subjects who completed treatment were 51.8±0.8 years old. They had the following alleles: A1+, n=22; A1−, n=23. There was no significant difference in the ages of these A1+ (51.4±0.9 years) and A1− (52.2±1.4 years) allelic subjects (F(1,43)=0.27, P=0.61).

As indicated earlier, 20 of the initial subjects dropped out of the study for a variety of reasons. The baseline GHQ total score of these subjects was 44.8±3.7. In the remaining 45 subjects the GHQ total score was 48.0±2.5. There was no significant difference in the GHQ total score between those who dropped out and those who remained in the study (F(1,63)=0.55, P=0.46).

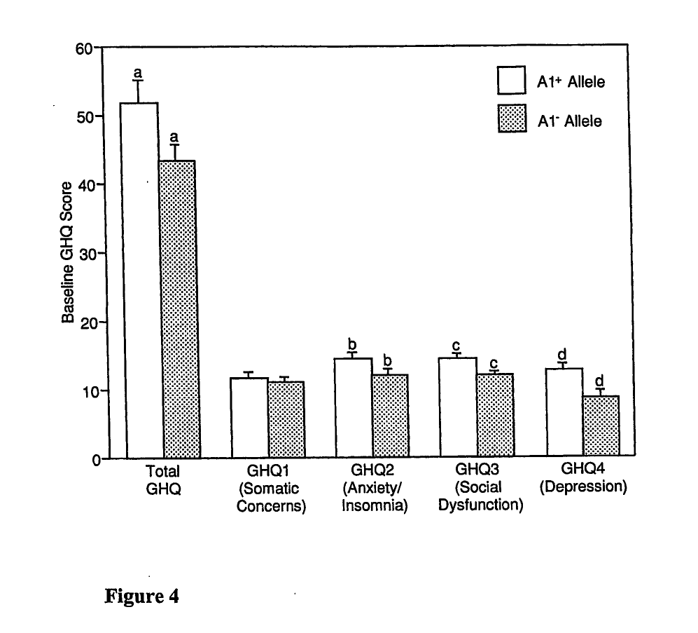

FIG. 4 shows the baseline GHQ total and subscale scores of the 65 patients who entered the paroxetine treatment study based on the presence or absence of the DRD2 A1 allele. GHQ total score was significantly higher in A1+ compared to A1− allelic patients. No significant difference was found in GHQ1 (somatic concerns) subscale score between these two allelic groups. However, GHQ2 (anxiety/insomnia), GHQ3 (social dysfunction) and GHQ4 (depression) subscale scores were significantly higher in A1+ allelic subjects when compared to their respective A1− allelic counterparts.

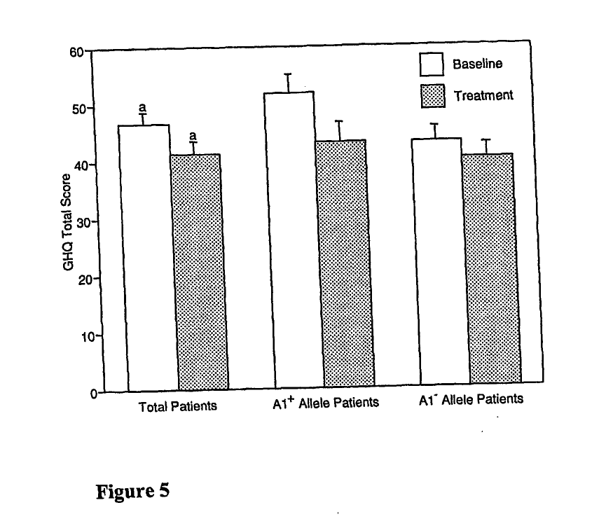

FIG. 5 shows the baseline and treatment GHQ total scores of the 45 patients who remained in the study. A significant improvement was found in the total patients during the course of paroxetine treatment (P=0.014). FIG. 5 also presents the baseline and treatment GHQ total scores of those patients based on their allelic status. The results showed no significant improvement in the GHQ total score in either A1+ or A1− patients.

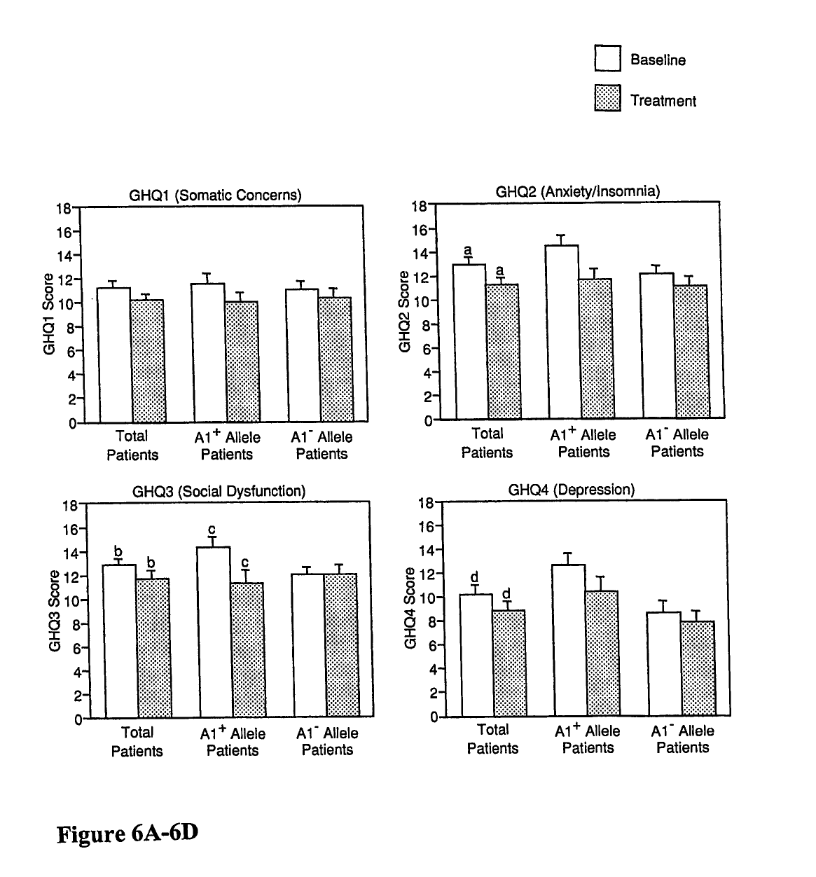

FIGS. 6A-6D present the results of the four GHQ subscale scores at baseline and at treatment of the 45 patients who remained in treatment, based on their allelic status. In the total patient group, GHQ1 subscale score was not significantly reduced over the course of treatment GHQ2 subscale score was significantly reduced in the total patient group over the course of treatment. There was a significant improvement in GHQ3 subscale score in the total patient group over the course of treatment, with A1+ allelic subjects experiencing a significant improvement. In contrast, A1− allelic subjects showed no such improvement. Finally, GHQ4 subscale score revealed a significant improvement in the total patient group during treatment.

Whereas, as shown in FIG. 4, significantly higher baseline GHQ total score and its three subscale scores (GHQ2, GHQ3, GHQ4) were found in the A1+ compared to the A1− allelic groups, at the end of Paroxetine treatment, no significant differences were found between these allelic groups in any of the GHQ scores measured: GHQ total (F(1,43)=0.001, P=0.99); GHQ1 (F(1,43)=1.5, P=0.23); GHQ2 (F(1,43)=0.14, P=0.71); GHQ3 (F(1,43)=1.1, P=0.30); GHQ4 (F(1,43)=2.5, P=0.12).

Discussion

In the present study, and consistent with previous studies (Marshall et al., 1998; Zygront et al., 1998), paroxetine treatment was effective in reducing psychopathological symptoms in Vietnam veterans with combat-related PTSD. However, this reduction in total symptoms measured was dependent on the patients' DRD2 allelic status, with DRD2 A1 allelic patients showing a significant reduction, while no such reduction was found in patients without this allele.

At baseline, total psychopathology score was greater in patients with than in patients without the DRD2 A1 allele. This initial greater total symptoms score in DRD2 A1 allelic patients was also evident in the subscale scores of anxiety/insomnia, social dysfunction and depression. However, after 8 weeks of paroxetine treatment, these subscale scores, like the total psychopathology score, were more significantly reduced in patients who carried the DRD2 A1 allele than those who did not. This resulted in no significant differences at the end of treatment between these two allelic groups in any of the GHQ scores measured.

Why do PTSD patients with the DRD2 A1 allele receive the greatest benefit after treatment with paroxetine? The answer to this question remains unknown. However, it may be hypothesized that baseline brain D2 dopamine receptor density could differentially affect subjects' drug response. In support of this hypothesis are studies which showed that paroxetine's antidepressant response is greater in subjects with low D2 dopamine receptor responsivity than subjects with high responsivity (Healey and McLean, 2000). Moreover, D2 dopamine receptor binding is lower at baseline in SSRI treatment depressed responders than in depressed non-responders (Klimke et al., 1999). This differential response extends to another drug (Volkow et al., 1999). In that PET study, the response to methylphenidate, a psychostinulant, was found to be pleasant and positively reinforcing in subjects with low brain D2 dopamine receptor levels, whereas those with high D2 dopamine receptor levels found this drug to be aversive.

Growing evidence (Noble et al., 1991; Thompson et al., 1997; Pohjalainen et al., 1998; Jönsson et al., 1999) suggests that individuals with the DRD2 A1 allele have reduced number of brain D2 dopamine receptors. In a double blind bromocriptine (a D2 dopamine receptor agonist)—placebo study of alcoholics (Lawford et al., 1995), the greatest decrease in anxiety and craving as well as the best retention rate was found in DRD2 A1 allelic patients who received bromocriptine. This pharmacogenetic approach to treatment is similar to the present study where paroxetine treatment was found to be most effective in reducing psychopathological symptoms in patients with the DRD2 A1 allele. Moreover, albeit not achieving a significant level, patient retention rate was greater (due to fewer adverse events) in patients who carried than those who did not carry the DRD2 A1 allele.

The reason why subjects with low D2 dopamine receptor binding respond to SSRIs remains unknown. However therapeutic actions of drugs and their adverse effects are thought to be due to neurotransmitter activating genes in target neurons (Stahl, 1999).

Fluoxetine causes an increase in nucleus accumbens shell D2 dopamine receptor mRNA resulting in increased D2 dopamine receptor postsynaptic binding in the nucleus accumbens (Ainsworth et al., 1998). Similarly, the SSRI citalopram increases D2 dopamine receptor mRNA in the striatum and nucleus accumbens (Dziedzicka-Wasylewska et al., 1997). This effect of citalopram on increased transcription of the D2 dopamine receptor gene is likely to be mediated by increased serotonin levels induced by SSRIs, as 5 hydtoxytryptophan also increases D2 dopamine receptor mRNA transcription (Kameda et al., 2000). Induction of D2 dopamine receptor gene expression way be the mechanism by which SSRIs reduce both anxiety and depression. Low pretreatment D2 dopamine receptor density may be a requirement for effective treatment. Conversely high pretreatment D2 dopamine receptor density may result in treatment resistance.

SSRI medications have similar efficacy in the treatment of depression and anxiety disorders. There is a positive response to SSRIs in subjects who have anxiety disorders without depression (de Boer et al. 1995), depression without anxiety (Nelson, 1999) and mixed anxiety and depressive disorder (Kara et al., 1994). There is also a significant association between anxiety and depressive scores in normal subjects and in patients with anxiety and depressive disorders (Kaneda and Fujii, 2000). With regard to genotypic structure, there is considerable evidence that anxiety and depression share a common genetic diathesis (Mineka et al., 1998). That common genetic diathesis may be the DRD2 gene is supported by the association of DRD2 variants with anxiety and depression, irrespective of the clinical disorder studied (Peroutka et al., 1998; Samochowiez et al., 2000). The impact of Paroxetine on both anxiety and depression scores in DRD2 A1 allelic subjects of the present study further argues for the interrelated nature of anxiety and depressive disorders in these PTSD patients.

The study is an open label trial and hence the influence of both patient and clinician expectancy cannot be controlled. However, as both patient and clinician were blind to the patient's DRD2 allelic status, the results are unlikely to be biased by expectancy effects. The sample size is modest, however, the significance of the results indicates a large effect size. The subjects were all males and the relevance of the findings to females remains unknown. Given these limitations, it is recommended that future studies employ double blind paroxetine-placebo trials using a larger number of patients than herein and consisting of both males and females.

In sum, PTSD patients with the DRD2 A1 allele, in contrast to those without this allele, showed a significant positive response to paroxetine treatment. The study suggests a pharmacogenetic approach to the treatment of PTSD.

### Example 4

### The A1 Allele of the D2 Receptor is Associated with Comorbid Depression and Anxiety in Untreated Veterans with Combat-Induced Post Traumatic Stress Disorder

A total of 57 untreated Caucasian Vietnam veterans with combat-induced post traumatic stress disorder (PTSD) were administered the General Health Questionnaire-28. Cluster analysis of these data identified two primary clusters. The first cluster, a high psychopathology cluster featured high comorbid levels of somatic concerns, anxiety, depression and social dysfunction. The low psychopathology cluster showed the reverse pattern. DRD2 A1+ (A1/A1, A1/A2 genotypes) veterans were significantly more likely to be found in the high psychopathology cluster (χ2=9.011, p<0.0003). There were no significant differences between clusters in alcohol consumption. The results indicate that DRD2 variants are associated with comorbid psychopathology in PTSD. Implications of this finding regarding both the underlying pathophysiology and treatment of mixed anxiety depressive disorder (cothymia) are discussed.

Introduction

Combat-related posttraumatic stress disorder (PTSD) is a highly debilitating condition with a chronic course. The quality of life of PTSD patients is frequently compromised by comorbid conditions such as social anxiety disorder, panic disorder, generalized anxiety disorder, dysthymia and major depressive disorder (Zatzick et al., 1997; O'Toole et al., 1998). PTSD does not develop in all persons subjected to traumatic stress suggesting considerable individual differences in susceptibility to this disorder (Stein et al 2002) and a genetic influence on symptoms remains even after accounting for combat exposure. Genetic factors contribute 13-30% of the variance in liability for re-expetiencing symptoms, 30-34% of avoidant symptoms and 28-32% of hyperarousal symptoms (True, et al 1993). Risk of developing PTSD following combat trauma in Vietnam was increased by a family history of paternal depression. In addition, pre-existing conduct disorder, panic disorder or generalised anxiety disorder and major depression were PTSD risk factors (Koenen et al., 2002). Individuals with PTSD report family psychiatric histories that closely resemble those found in sufferers of coexisting anxiety and depression. However, patients diagnosed with anxiety disorders alone and control participants without psychiatric disorder report dissimilar family histories (Reich et al 1996). Importantly family history of psychiatric illness is found more frequently in combat veterans with comorbid depression and PTSD compared with those with PTSD alone. Stressor severity and number of traumatic events were not associated with comorbid depression (Kozaric-Kovacic, Hercigonja & Grubisic-Ilic, 2001).

A large twin study of 4072 male-male Vietnam veteran twins revealed that alcohol use and PTSD symptoms are associated and share a common genetic aetiological vulnerability (McLeod et al 2001). Combat veterans with PTSD carrying the A1 allele of the DRD2 (A1/A1 or A1/A2; A1+) consume twice as much alcohol at twice the rate of those without this allele (A2/A2 or A1−) (Young et al 2002). Previous studies have found an inconsistent association between DRD2 variants and PTSD but have not examined the confound of possible substance misuse in the populations studied (Comings et al, 1996; Gelernter et al, 1999). As liability for alcohol problems and PTSD symptoms share a common genetic vulnerability and A1+ veterans have increased alcohol consumption we investigated whether differences in PTSD symptomatology was associated with A1+ status. Additionally, as PTSD subjects have family histories which are similar to those found in mixed anxiety depression we investigated whether co morbid anxiety, depression, social dysfunction and somatic concerns was associated with A1+ individuals with PTSD.

Method

Fifty-five unrelated male Caucasian patients diagnosed with PTSD who were presenting for treatment were recruited for study. All subjects were Vietnam combat veterans who had served in the Australian armed forces. None were being treated with psychotropic medication. Patients were excluded from the study if they had a diagnosis of psychosis, bipolar disorder, obsessive compulsive disorder, or organic brain syndrome including dementia. All subjects had sufficient comprehension of English and could understand the administered questionnaires.

Patients were assessed for PTSD by a consultant psychiatrist (BL) or a senior psychiatric registrar (BK) using Diagnostic and Statistical Manual of Mental Disorders (DSM-IV) criteria. PTSD diagnosis was confirmed for all subjects. Furthermore, every patient exceeded the clinical cut-off score of 94 on the Mississippi Scale for combat-related posttraumatic stress disorder (Zatzick et al., 1997). The validity and reliability of the Mississippi scale are well established in veterans (Keane et al., 1988). Patients then underwent clinical history taking by a psychiatrist or by a clinical nurse. Demographic data and ethnic background were also obtained.

The General Health Questionnaire-28 (GHQ) was administered. The GHQ-28 measures four psychopathological factors particularly relevant to PTSD and comorbid psychiatric conditions: somatic concerns (GHQ1), anxiety/insomnia (GHQ2), social dysfunction (GHQ3) and depression (GHQ4). The GHQ has been widely validated internationally as a means of detecting psychiatric caseness and is sensitive to change (Ormel et al., 1989). The GHQ has utility as a follow-up measure of veteran mental health following exposure to combat (Deahl et al., 1994) and is sensitive to changes in combat-related PTSD symptoms (Ward, 1997) and has been used to assess symptom severity (Fuesner et al 2001, Lawford et al, in press).

All participants provided written informed consent and were able to terminate the study without prejudice. Institutional ethics approval was obtained from the Greenslopes Private Hospital.

Genotyping. A 10 ml blood sample was drawn from each patient. Genomic DNA was extracted employing standard techniques and used as a template for determination of TaqI A DRD2 alleles by the polymerase chain reaction (Grandy et al., 1993). The amplification of DNA was carried out using a Perkin Elmer GeneAmp 9600 thermocycler. Approximately 500 ng of amplified DNA was digested with five units of TaqI restriction enzyme (New England Biolabs) at 65° C. overnight. The resulting products were separated by electrophoresis in a 2.5% agarose gel containing ethidium bromide and visualized under ultraviolet light. Three genotypes are obtained: the A1A2 genotype is revealed by three fragments: 310 bp, 180 bp and 130 bp. The A2A2 genotype by two fragments: 180 bp and 130 bp. The A1A1 genotype is shown by the uncleaved 310 bp fragment. A1+ allelic subjects are those that either have the A1A1 or A1A2 genotype; A1− allelic subjects have the A2A2 genotype only.

Information coded from the interviews, GHQ-28, Mississippi Scale and genotyping results were entered into a computer database. Nominal data were analysed by Yates corrected X² test and continuous data were analysed by analysis of variance. A joining tree cluster analysis was employed to identify various symptom cluster groups (Everitt et al 2001). P values≦0.05 were considered statistically significant.

Results

The mean age of the 57 patients who entered treatment was 51.2 years. They had the following genotypes: A1A2 (A1+ allele), n=22; and A2A2 (A1− allele), n=35. This genotype distribution did not deviate from the Hardy-Weinberg equilibrium (χ²=2.86, P=0.091). There was no significant difference in the ages of A1+ (50.8±0.7 years) and A1− (51.5±1.0 years) allelic subjects (F(1,55)=0.23, P=0.64).

The GHQ data were analysed using joining tree cluster analysis in order to determine subgroups of patients that differed according to symptom profile. These analyses were conducted blind to allelic status. The analysis showed four clusters and cluster scores for each of these are presented in Table 2. Cluster 1 (n=32) was characterised by low scores on all four GHQ factors, Cluster 2 (n=3) was characterised by low scores on somatic symptoms and social dysfunction, moderate scores on anxiety but significant depressive symptomatology. Cluster 3 (n=16) was characterised by raised scores on all GHQ factors and Cluster 4 (n=6) showed significant somatic and anxiety symptoms with low scores on social dysfunction and an absence of depressive symptoms. Only two clusters, Cluster 1, the “low psychopathology” cluster and Cluster 3, the “high psychopathology” cluster had sufficient subject numbers for further statistical analysis. FIG. 7 presents mean Mississippi scores of Cluster1 and Cluster 3. Analysis of variance revealed Mississippi scores to be significantly higher in Cluster 3 when compared to Cluster 1 (Cluster 1, mean MISSI=121.3, s.d.=17.9, Cluster 3, mean MISSI=143.3, s.d.=13.5, F (1,45)=18.75, p<0.0001) (see FIG. 7) However there was no significant difference in alcohol consumption in grams per day between Cluster 1 (mean=73.98 grams per day, s.d.=124.89) and Cluster 3 (mean=131.75 grams per day, s.d=140.56) (F(1,45)=2.07, p=0.16). In the “low psychopathology” Cluster 1, 8 patients were A1+ and 24 patients were A1−. The “high psychopathology” Cluster 3 contained 12 patients who with A1+ status and 4 patients who with A1− status. Yate's corrected Chi-square statistic revealed that Cluster 3 had a significantly higher prevalence of A1+ allelic participants than Cluster 1(χ2=9.011, p<0.003)

TABLE 2

| | GHQ1 | GHQ2 | GHQ3 | |
|---|---|---|---|---|
| | (somatic | (anxiety | (social | GHQ4 |
| Cluster | symptoms) | symptoms) | dysfunction) | (depression) |
| 1 (n = 32) | −0.581 | −0.767 | −0.516 | −0.525 |
| 2 (n = 3) | −0.129 | 0.288 | −0.788 | 1.331 |
| 3 (n = 16) | 0.803 | 1.153 | 1.131 | 1.115 |
| 4 (n = 6) | 1.023 | 0.87 | 0.120 | −0.846 |

Discussion

The major finding of this paper was the strong association of A1+ status with comorbid somatic symptoms, anxiety, social dysfunction, and depression which was independent of alcohol effects. Furthermore, the identification of two main symptom clusters confirms a previous study (Hallman et al 2003)of 1161 Gulf War veterans where cluster analysis again identified two primary groups. The first group reported good health and few moderate or severe symptoms. The second group was characterised by fair or poor health and increased frequency of moderate/severe symptoms.

Epidemiological studies and clinical experience show that the comorbidity of anxiety and depressive disorders is frequent (Bjelland, & Dahl, 1999). These observations have led to the proposed establishment of a new disorder of mixed anxiety and depression (MAD) (Barlow & Campbell, 2000). MAD or cothymia has a significantly worse outcome than either an anxiety or depressive diagnosis alone. Furthermore, longitudinal data reveal social functioning in addition to anxiety and depression, is significantly worse in patients with cothymia compared to those with either anxiety or depression alone Tyrer et al, 2001).

The association of A1+ individuals with comorbid depression/anxiety/social dysfunction/somatic concerns implicates the involvement of D2 receptors in the coexistence of these disorders. There is compelling evidence that A1+ individuals have a reduced density of brain D2 dopamine receptors (Noble et al, 1991, Thompson et al, 1997, Pohjalainen et al, 1998,Jonsson, et al, 1999). An early brain autopsy study (Noble et al., 1991) found a significant reduction of approximately 30% in the number of D2 dopamine receptors (Bmax) in the caudate nucleus of A1+ compared to A1− allelic subjects. Moreover, a significant progressive decline in Bmax was found across A2/A2, A1/A2, A1/A1 genotypes in that order. There was no difference in D2 dopamine binding affinity (Kd) between A1+ and A1− allelic subjects. Thompson et al., 1997 also reported 30%-40% reduction in D2 dopamine receptor density in the striatum of A1+ individuals compared to A1− individuals. An in-vivo study of healthy Finnish volunteers (Pohjalainen et al., 1998) showed significantly decreased D2 receptor density in the striatum of A1+ when compared with A1− allelic subjects. Again, there was no difference in the Kd between the two groups. In Jonsson et al (1999), another PET study of healthy humans using (11C) raclopride, a significant association of the A1 allele with low D2 dopamine receptor density was found. While, Laruelle et al (1998) reported no association between reduced D2 binding and the A1 allele subjects an editorial (Hitzemann, 1998) suggested the study had insufficient power to detect a significant difference between A1+ and A1− individuals.

The most likely explanation of the association of the A1 allele, a polymorphism in a non-coding region of the DRD2 gene, with reduced D2 receptor density, is that the sequence variation causing the Taq1 A polymorphism is in linkage disequilibrium with functional allelic variants that affect receptor expression Noble, et al, 1998, Comings et al, 1991, O'Hara et al 1993, Arinami et al, 1997). The Taq 1A variants are now known to be in linkage disequilibrium with C957T, a synonymous mutation in the human DRD2. Furthermore, C957T affects mRNA folding leading to both less stable mRNA and decreased translation. These effects dramatically diminish dopamine induced up-regulation of D2 receptors (Duan et al, 2003).

Both animal and human research implicate decreased D2 receptor functioning in the aetiology and treatment of depression and anxiety. Animal studies have reported that decreased caudate D2-like receptor functioning is associated with learned helplessness which is a generally recognised model of depression (ram et al 2002). Morphine can reverse a state of learned helplessness in rats and haloperidol, a D2 receptor antagonist, blocks this action suggesting D2 receptor mediation of learned helplessness (lesson et al 1999). Social subordination in female cynomolgus monkeys is also associated with decreased D2 receptor function (Shively, 1998).

There are similar data regarding the role of the D2 receptor in the genesis of anxiety. In the rat amygdala both D1 and D2 receptors are involved in the mediation of the startle response (Greba, Gifkins & Kokkinidis, 2001). The action of quinpirole, a D2 agonist, has confirmed that D2 dopamine receptors are involved in social and emotional activity in mice (Gendreau et al, 1998). Microinfusion of quinpirole suppresses the fear arousing properties of foot shock in rats (Gifkins et al, 2002). Dopamine D2 autoreceptor agonists inhibit ultrasonic vocalisation in rats reflecting an anxiolytic effect in these animals Bartoszyk, 1998).

Social phobia patients have low binding of [(123)I]IBZM to striatal D(2) receptors (Schneier et al, 2000). Furthermore there is strong linkage between the A1 allele for the D(2) receptor and the Tridimensional Personality Questionnaire (TPQ) harm avoidance scale. This scale largely reflects introversion (worry, pessimism and shyness) (Hill et al, 1999). Dopamine system hypoactivity has also been linked to trait detachment, social anxiety disorder and diminished reward and incentive function (Schneier et al, 2002).

There is a significant relationship between low dopamine D2 receptor binding and NEO Personality Inventory-Revised non-clinical personality trait of depression (Kestler et al, 2000). Depressed patients exhibit a hypersensitive response to dextroamphetamine with severity of depression correlating highly with the rewarding effects of dextroamphetamine (Tremblay et al, 2002). Subjects administered intravenous methylphenidate experience reward that is inversely proportional to D2 receptor binding (Volkow et al., 1999, 2002).This suggests that severe depression is associated with decreased brain D2 density.

Schizophrenics treated with typical antipsychotic drugs show worsening of depressive symptoms with increasing D2 receptor occupancy (Bressan et al, 2002). Similarly, a further PET study of patients with schizophrenia treated with the a-typical antipsychotics olanzapine or risperidone demonstrated that D2 receptor occupancy was proportional to subjective experience of depression (de Haan et al, 2000). Furthermore, growth hormone response to apomorphine, a D2 agonist, is reduced in depressed patients who commit suicide reflecting reduced dopaminergic activity. (Pitchot et al, 2001).

The pharmacotherapeutic treatment of depression and anxiety states is similar. Selective serotonin reuptake inhibitors (Berk, 2000, Rausch et al, 2001) and venlafaxine (Gorman and Papp, 2000) are effective in mixed anxiety and depression. The therapeutic actions of these drugs and their adverse effects are due to neurotransmitter activating genes in target neurons (Stahl, 1999). Fluoxetine causes an increase in nucleus accumbens shell D2 dopamine receptor mRNA resulting in increased D2 dopamine receptor postsynaptic binding in the nucleus accumbens (Ainsworth et al., 1998). Similarly, the SSRI citalopram increases D2 dopanine receptor mRNA in the striatum and nucleus accumbens (Dziedzicka-Wasylewska et al., 1997). This effect of citalopram on increased transcription of the D2 dopamine receptor gene is likely to be mediated by increased serotonin levels induced by SSRIs, as 5 hydroxytryptophan also increases D2 dopamine receptor mRNA transcription (Kameda et al., 2000). Dopamine D2 receptor binding increases in SSRI treatment responders and decreases in non responders (Klimke et al, 1999). suggesting that induction of D2 dopamine receptor gene expression is likely to be an important mechanism by which SSRIs exert therapeutic effects.

Dopamine receptor ligands also have both anxiolytic and antidepressant effects (Bartoszyk, 1998, Theohar 1982)). A double-blind placebo controlled study (Lawford et al, 1995) examined the effects of a D2 dopamine receptor agonist, bromoctiptine (BRO) and placebo (PLA) on treatment outcome in alcoholism. The results showed that in the four groups of alcoholics studied (BRO A1+, BRO A1−, PLA A1+, PLA A1−) the greatest and most significant decreases in craving and anxiety were found in A1+ alcoholics treated with bromoctiptine (BRO A1+). Bromoctiptine has similar efficacy as imipramine and amitriptyline for the treatment of endogenous depression (Bouras, Bridges et al, 1982 & Waehrens & Gerlach, 1981, Theohar et al, 1982).

Considerable evidence from both animal and human studies emphasises the importance of low D2 receptor functioning in both anxiety and depression. It is not therefore surprising that the A1 allele for the D2 receptor that is genotypically expressed as reduced D2 density is associated with comorbid severe anxiety and depression in combat veterans with PTSD. Further investigation of this polymorphism in anxiety/depressive disorders may improve understanding of the pathophysiology of these states and eventually lead to targeted pharmacogenetic treatment interventions.

### REFERENCES

Ainsworth, K, et al. 1998. Psychopharmacology 140, 470-477.
Arinami T, et al. (1997) Human Molecular Genetics 6:577.
Aropax® (Paroxetine) product information, 1999. SmithKline Beecham (Aust) Pty Ltd, Item number 92536.
Ballinger, J. C., et al. 2000. J. Clin. Psychiatry 61, Supp 5, 60-66.
Barlow, D H, Campbell, L. A, 2000. Compr Psychiatry. 41(2 Suppl 1) 55-60.
Bartoszyk, G D, 1998. LifeSci 62:649-63
Basturk M, et al. (2001) Prog Neuropsychopharmacol Biol Psychiatry. 25;315-22
Ben-Jonathon N & Hnasko, R (2001). Endocr Rev. 6, 724-63.
Berk, M, 2000. Int Clin Psychopharmacol. 15 Suppl 2: S41-5.
Besson A, Privat A M, Eschalier A, Fialip J 1998. Pharmacol Biochem Behav 60:519-25
Bjelland, I, Dahl, A A, 1999. Tidsskr Nor Laegeforen. 119: 3277-80.
Bouras, N, Bridges, P K. 1982. Curr Med Res Opin 8:150-153.
Brady, K., et al. 2000. JAMA 12:283, 1837-1844.
Bressan, R A, et al. 2002. Schizophr Res. 56: 31-6.
Broich K et al. (1998). Pharmacopsychiatry: 31;159-62
Chounaird G, et al. (1980) Can J Neurol Sci 7:233
Comings D E, et al. (1991) Journal of the American Medical Association. 266:1793-1800
Comings, D E, et al. (1996). Biol Psychiatry, 40, 386-72.
David S R, et al. (2000) Clin Ther; 9:1085-96.
De Duyn P P, Wirshung W C. (2001) J Clin Psychiatry;62 Suppl 21:19-22
de Haan, L, et al. 2000. Am J Psychiatry 57:1019-20.
Deahl, M. P., et al. 1994. Br. J. Psychiatry 165, 60-65.
Delgado, P. L., 2000. J. Clin. Psychiatry, 61 Suppl 6, 7-11.
den Boer, J. A., et al. 1995. Int. Clin. Psychopharmacol. Suppl 4, 47-52.
Deutch, A. Y., Roth, R. H., 1990. Prog. Brain Res. 85, 357-393.
Deutch, A. Y., Young, C. D., 1995. In:, Friedman, M. J., Charney, D. S., Deutch, A. Y. Eds.), Neurobiological and Clinical Consequences of Stress. Lippincott-Raven Press, Philadelphia, pp. 163-176.
Dow, B., Kline, N., 1997. Ann. Clin. Psychiatry 9, 1-5.
Duan J, et al. (2003) Human Molecular Genetics 12:205-216
Dziedzicka-Wasylewska, M., et al. 1997. J. Neural. Transm. 10, 515-524.
Everitt B S, Landau S, Leese M;. 2001. Cluster Analysis. London; Edward Arnold
Fuesner, J, et al. (2001). Psychiatry Research, 104, 109-17.
Gelernter, J, et al. (1999). Biol Psychiatry, 45, 620-5.
Gendreau, P L, et al. (1998). Neuropsychopharmacology, 18, 210-21.
Gerlach J, 2002 Ann Clin Psychiatry 14; 47-57
Gifkins, A, et al. (2002). Behav Neuorsci 116: 785-94
Gobert, A., et al. 2000. Synapse 36, 205-221.
Gorman J M, Papp L A. 2000 Depress Anxiety. 12 Suppl 1:77-80.
Grandy D K, et al. (1993) Hum Mol Genet 2, 2197
Greba Q, et al. (2001). Brain Research, 899, 218-26.
Grevle L, et al. 2000. Mov Disord; 15:1070-4
Halbreich U, et al. (2003) Psychoneuroendocrinology, Suppl 1, 53-67.
Hallman W K, et al. 2003 Am. J. Public Health; 93:624-630
Healy, E., McKeon, P., 2000. J. Psychopharmacology 14, 152-156.
Heck A H, et al. 2000 Schizophr Res; 46:97-105
Hidalgo, R. B., Davidson, J. R. T., 2000. J. Psychopharmacol. 14, 70-76.
Hill, S Y, et al. 1999. Am J Med Genet 88:634-41.
Hitzemann R J, (1998) Mol Psychiatry, 3:198-203
Hugues F C, et al. (2000). Ann Med Interne. 151;10-7
Iaccarino C, et al. (2002) Proc. Natl. Acad. Sci.; 99:14530-5
Jarvinen A, et al. (1992) Neutopeptides; 21:183-91
Jauss M, et al. (1998) Pharmacopsychiatry; 4:146-8
Jeste D V, et al. 1999. J Am Geriatr Soc; 47:716-9
Jonsson E G, et al. (1999) Mol Psychiatry, 4:290-6
Kameda, K., et al. 2000. J. Mol. Neuorsci. 14, 77-86.
Kaneda, Y., Fujii, A., 2000. J. Med. Invest. 47, 1418.
Kapur S, Remington G. (2001a) Biol Psychiatry 50:873-83
Kapur S, et al. (2001b) Am J Psychiatry; 159:255-262
Kapur S, et al. (2000) Am J Psychiatry; 4:514-20
Kata, S., et al. 2000. Psychiatry Res. 94, 59-66.
Keane, T. M., et al. 1988. J. Consult Clin. Psychol. 56, 85-90.
Keltner N L, et al. (2002) Perspect Psychiatr Care 38:111-6
Kestler, L P, et al. 2000. Neuropsychiatry Neutopsychol Behav Neurol 13:48-52.
Klimke, A., et al. 1999. Psychiatry Res. Neuroimaging. 90, 91-101.
Koenen K C, et al. 2002 J Nerv Ment Dis; 190:209-218
Kopala L C, 1997. Int Clin Psychopharmacol; 12 Suppl 4:S11-8
Kozaric-Kovacic D, et al. (2001). Croat Med J, 42, 165-70.
Kram M L, et al. (2002) Prog Neuropsychopharmacol Biol Psychiatry; 26:639-45
Laruelle M, et al. (1998) Mol Psychiatry 3:261-5
Lavalaye J, Linszene D H, Booij J, Reneman L, Gersons B P, van Royen E A(1999). Dopamine D2 receptor occupancy by olanzapine or risperidone in young patients with schizophrenia. Psychiatry Research, 8, 33-44.
Lawford, B. R., et al. 1995. Nature Med. 1, 337-341.
Love R C, Nelson M W. 2000. Expert Opin Pharmacother; 1:1441-53
Maguire G A (2002). J Clin Psychiatry, 63 Suppl 4, 56-62.
Markianos M, et al. (2002) Schizophrenia Res.; 56:11-7
Markianos M, et al. (2001) Eur Arch Psychiatry Clin Neurosci 251:141-6
Marshall, R. D., et al. 1998. J. Clin. Psychopharmacol. 18, 10-18.
McCreadie R G, Macdonald I M. (1997) Br J Psychiatry, 131:310-6
McLeod, D. S, et al. (2001). J Traum. Stress. 14, 259-75.
Mendlin, A., et al. 1998. Eur. J. Pharmacol. 351, 291-298.
Mihara K, et al. (2000) Psychopharmacology (Berl) 149: 246-50
Mihara K, et al. (2001) Am J Med Genet; 105:271-4
Mineka, S., et al. 1998. Annu. Rev. Psychol. 49, 377-412.
Nelson, J. C., 1999. Biol. Psychiatry 46, 1301-1308.
Ng, N.-K., et al. 1999. J. Neurosci Res. 55, 600-607.
Noble E P, et al. (1991) Arch Gen Psychiatry, 48:648-54
Noble E P, et al. (1994) Int J Eat Disord, 15, 205-17
Noble, E P, et al. (1998). Psychiatry Research, 81, 133-147.
Nordstrom A L, et al. (1993). Biol Psychiatry:33: 227-235
Nyberg S, et al. 1999. Am J Psychiatry; 156:869-75
O'Hara, B F, et al. (1993). Hum Hered 43: 209-218
O'Toole, B. I., et al. 1998. Aust N. Z. J. Psychiatry, 32, 32-42.
Ormel, J., et al. 1989. Psychol. Med., 19, 1007-1013.
Otani S, et al. 1999. J Neurosci, 19, 9788-802.
Petty R G. (1999) Schizophr Res 35 Suppl. S67-73.
Pitchot, W, et al. 2001. Psychoneuroendocriology. 26:331-5.
Pohjalainen T, et al. (1998) Mol Psychiatry, 3:256-60
Pohjalainen, T., et al. 1998. Mol. Medicine 4, 14-21.
Rausch, J L, et al. 2001. J Clin Psychopharmocol. 21: 139-42.
Reich J, et al. (1996). Acta Psychiatrica Scand, 93, 105-12.
Remington G, Kapur S, (2000) Psychopharmacology(Berlin); 148:3-15.
Rollema, H., et al. 2000. Biol. Psychiatry 48, 229-237.
Samochowiec, J., et al. 2000. Pharmacogenetics 10, 471-475.
Schneier, F R, et al. 2002. Psychiatr Clin North Am 25: 757-74
Schneier, F R, et al. 2000. Am J Psychiatry 58:237-28
Shively C A (1998). Biol Psychiatry, 44, 882-91.
Silvestri, S., et al. 2000. Psychopharmacology 152, 174-180.
Stahl, S. M., 1999. J. Clin. Psychiatry 60, 813-814.
Stein M B, et al. 2002 Am J Psychiatry 159:1675-81
Stein, M. B., et al. 1998. JAMA 280, 708-713.
Sussman K, 2002. Int Clin Psychopharmacol; 17 Suppl 3:S29-33
Tandon R, 2002. Psychiatr Q; 73:297-311
Thase, M. E., et al. 1996. Arch. Gen. Psychiatry 53, 777-784.
Theohar, C, et al. 1982. Arzneimittelforschung 32:783-787.
Thompson J, et al..(1997) Pharmacogenetics; 7:479-84
Tremblay, L K, et al. 2002. Arch Gen Psychiatry 2002. 59:409-16.
True W R, et al. (1993) Arch Gen Psychiatry 50;257-64
Tyrer, P, et al. 2001. Eur Arch Psychiatry Clin Neuorsci. 251 Suppl 2: 1153-6.
Van Houdenhove, B., et al. 1997. J. Int. Med. Res. 25, 340-353.
Vanderpump M, et al. (1998). Clinical Endocrinology, 48, 1365-2265.
Volavka, J, et al. (2002). Am J Psychiatry, 159, 255-62.
Volkow, N. D., et al. 1999. Am. J. Psychiatry 156, 1440-1443.
Volkow, N D, et al. (2002). Synapse, 46; 79-82
Waehrens, J, Gerlach, J. 1981. J Affect Disord 3:193-202.
Wang J et al. 2001. Neurology; 56:1757-9
Ward, W., 1997. Aust. N. Z. J. Psychiatry 31, 184-193.
Wilner, P., 1997. Int. Clin. Psychopharmacol. 12, Suppl 3, S7-S14.
Yamada Y, et al. (2002) Synapse: 46;32-7
Young, R M, et al. (2002). Alcohol Alcohol, 37, 451-6.
Zatzick, D. F., et al. 1997. Am. J. Psychiatry, 154, 1690-1695.
Zygmont, M., et al. 1998. J. Clin. Psychiatry 59, 241-245.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of identifying a candidate psychiatric patient for treatment with medication that acts at a D2 dopamine receptor or influences D2 dopamine receptor density, the method comprising:
determining whether the patient's DRD2 genotype is Taq1A allele positive (A1+) or Taq1A allele negative (A1−);
wherein the patient's DRD2 genotype provides a basis for selecting treatment.

2. The method of claim 1, wherein:
an A1+ genotype is indicative of a candidate for treatment with high dose high DRD2 binding atypical antipsychotics and/or SSRIs that increase D2 dopamine receptor density; and
an A1− genotype is indicative of a candidate for treatment with low dose high or low D2 dopamine receptor binding atypical antipsychotics or alternative antidepressant.

2. The method of claim 1, wherein the psychiatric patient suffers from schizophrenia.

3. The method of claim 1, wherein the patient suffers from post-traumatic stress disorder (PTSD), depression, social anxiety or mixed anxiety and depressive states.

4. The method of claim 1 when the patient suffers from Parkinson's disease.
